(19) United States
(12) Patent Application Publication
(10) Pub. No.: US 2013/0046367 A1
(43) Pub. Date: Feb. 21, 2013

(54) Lipid Removal Device for Treating Blepharitis (Meibomian Gland Dysfunction)

(71) Applicant: Willie Ying-Wei Chen, Taipei City (TW)

(72) Inventor: Willie Ying-Wei Chen, Taipei City (TW)

(21) Appl. No.: 13/212,313

(22) Filed: Aug. 18, 2011

(51) Int. Cl. A61F 7/08

(52) U.S. Cl. 607/113

(57) ABSTRACT

A lipid removal device for treating Meibomian gland dysfunction includes a housing defining a compartment. A thermally conductive coupler is mounted in a hollow seat formed on an end of the housing. A first end of the coupler has a coupling portion engaged with a needle to be inserted into a blocked Meibomian gland of an eyelid of a human body for melting and removing lipids blocking the Meibomian gland. A heating member is mounted in the compartment and connected to a second end of the coupler. A temperature sensor is mounted in the compartment and connected to the coupler. A control device is mounted in the compartment and electrically connected to the heating member and the temperature sensor. The control device cooperates with the temperature sensor to control a temperature of the needle to be in a preset temperature range by controlling on/off of an electric power source for the heating member.

### BACKGROUND OF THE INVENTION

The present invention relates to a lipid removal device for treating blepharitis (Meibomian gland dysfunction) and, more particularly, to a lipid removal device used in a surgical treatment option for blepharitis (Meibomian gland dysfunction).

Meibomian gland dysfunction (or posterior blepharitis) is a common eye disease in which the Meibomian glands in the eyelids are blocked by lipids. With the conventional treatment, only small percentage of patients has reached a long term success. In a recent survey, more the ⅓ of population over age of 50 suffer from Meibomian gland dysfunction along with dry eye. In a current method used in an operation for treating Meibomian gland dysfunction, a physician will insert a metal tube with a diameter of 0.2-0.4 mm into a blocked Meibomian gland to remove the lipids blocking the Meibomian gland.

Thus, a need exists for a novel device for enhancing the treating effect of Meibomian gland dysfunction.

### BRIEF SUMMARY OF THE INVENTION

A lipid removal device for treating Meibomian gland dysfunction according to the present invention includes a housing defining a compartment. The housing includes a hollow seat formed on an end of the housing. A thermally conductive coupler is mounted in the seat of the housing. A first end of the coupler has a coupling portion engaged with an end of a needle. The other end of the needle is adapted to be inserted into a blocked Meibomian gland of an eyelid of a human body and adapted to be heated to remove lipids blocking the Meibomian gland. A heating member is mounted in the compartment of the housing and connected to a second end of the coupler. A temperature sensor is mounted in the compartment of the housing and connected to the coupler. A control device is mounted in the compartment of the housing and electrically connected to a power cord of the heating member and to a transmission line of the temperature sensor. The control device cooperates with the temperature sensor to control on/off of an electric power source connected to the power cord, controlling a temperature of the needle to be in a preset temperature range.

Preferably, the housing includes two half housings engaged together to define the compartment. The electric power source is a battery received in a battery space of the compartment and electrically connected to the heating member. The housing further includes a lid for closing the battery space.

Preferably, the coupling portion of the coupler has a section extending out of the seat of the housing. The section of the coupling portion includes an engagement hole receiving and holding the end of the needle.

Preferably, the second end of the coupler includes two spaced receptacles respectively coupling with the heating member and the temperature sensor. A thermal insulating ring is mounted around the coupler and sandwiched between the coupler and an inner periphery of the seat, positioning the coupler in the seat.

Preferably, an enlarged shank is mounted on the end of the needle and has an outer diameter larger than that of the end of the needle. A protective cover is mounted around the other end of the needle and the shank.

Preferably, the coupling portion of the coupler includes a conic outer periphery tapering away from the second end of the coupler. The coupling portion includes a plurality of slits extending longitudinally. The seat of the housing includes an outer periphery having a threaded portion. A positioning cap has an inner threaded portion engaged with the threaded portion of the seat. The positioning cap includes a positioning portion having a central conical hole. The central conical hole has an inner periphery abutting the conic outer periphery of the coupling portion of the coupler to hold the end of the needle by the coupling portion. The other end of the needle extends out of the central conical hole.

Preferably, the control device includes a switch for controlling on/off of the electric power source for the heating member. The control device further includes a temperature adjusting member operable to adjust the preset temperature range of the needle. Preferably, the preset temperature range of the needle is between 40° C. and 45° C.

The present invention will become clearer in light of the following detailed description of illustrative embodiments of this invention described in connection with the drawings.

### DESCRIPTION OF THE DRAWINGS

FIG. 1 shows a perspective view of a lipid removal device for treating Meibomian gland dysfunction according to the present invention.

FIG. 2 shows an exploded, perspective view of the lipid removal device of FIG. 1.

FIG. 3 shows an exploded, perspective view of a portion of the lipid removal device of FIG. 1.

FIG. 4 shows a cross sectional view of the portion of the lipid removal device of FIG. 3 after assembly.

FIG. 5 shows an exploded, cross sectional view of the portion of the lipid removal device of FIG. 4.

FIG. 6 shows an exploded, perspective view of a needle and a protective cover of the lipid removal device of FIG. 1, with portions of the needle and the protective cover broken away.

FIG. 7 shows a view similar to FIG. 4, wherein the lipid removal device is in a state ready for use.

### DETAILED DESCRIPTION OF THE INVENTION

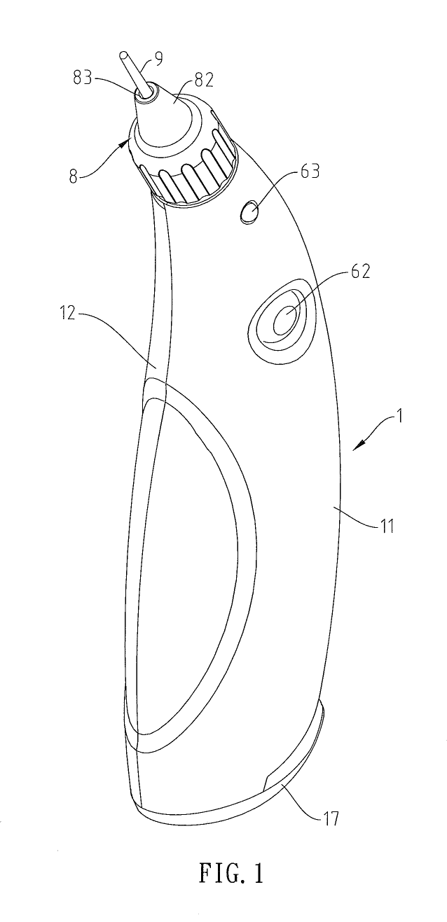

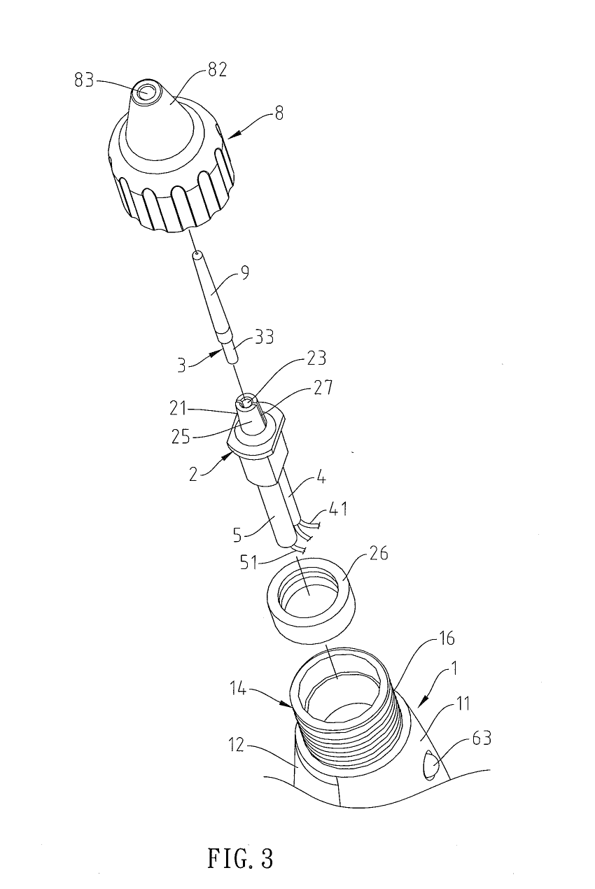

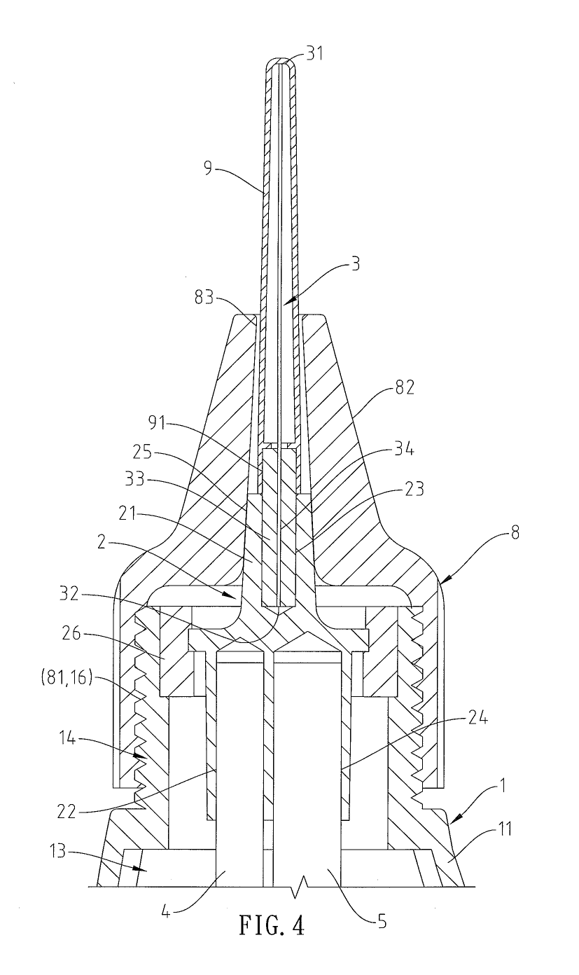

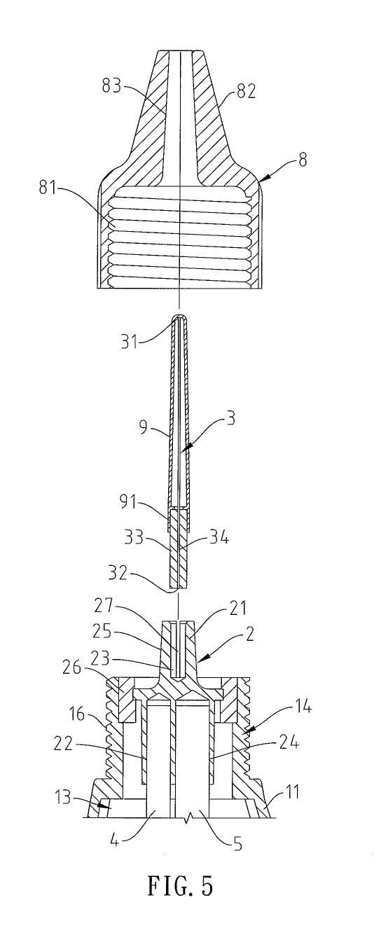

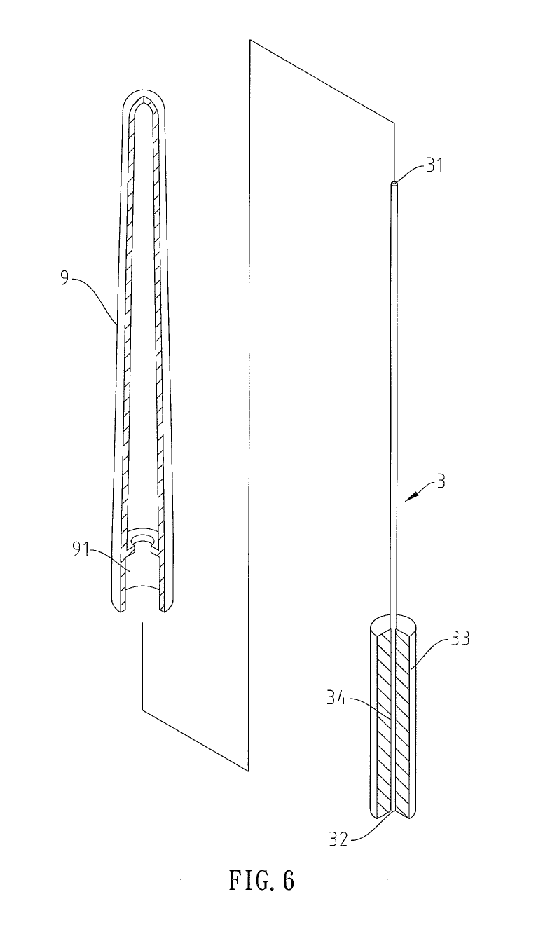

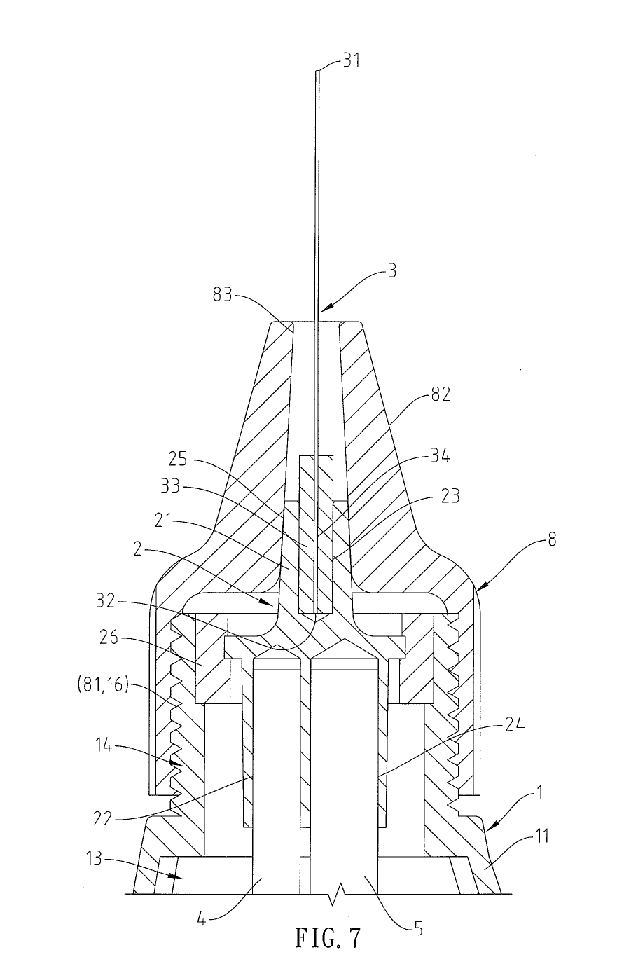

With reference to FIGS. 1-7, a lipid removal device for treating Meibomian gland dysfunction according to the present invention includes a housing 1 defining a compartment 13. The housing 1 includes a hollow seat 14 formed on an end of the housing 1. A thermally conductive coupler 2 is mounted in the seat 14 of the housing 1 and includes first and second ends. The first end of the coupler 2 has a coupling portion 21 engaged with an end 32 of a needle 3.

The needle 3 is made of metal and generally maintained at a temperature between 45° C. and 50° C. In use, a doctor holds the housing 1 and inserts the other end 31 of the needle 31 into a blocked Meibomian gland of an eyelid of a human body to melt and remove lipids blocking the Meibomian gland. The temperature of a cornea of an eye is normally 32.3±0.5° C. The temperature of the eyelid is normally 34.0±1.3° C. Skin burn occurs if the skin temperature reaches and exceeds 55° C. Thus, the temperature for melting the lipids is preferably in a range between 45° C. and 50° C.

With reference to FIGS. 1-5, the lipid removal device for treating Meibomian gland dysfunction further includes a heating member 4 mounted in the compartment 13 of the housing 1 and connected to the second end of the coupler 2. A temperature sensor 5 is mounted in the compartment 13 of the housing 1 and connected to the coupler 2. A control device 6 is mounted in the compartment 13 of the housing 1 and electrically connected to a power cord 41 of the heating member 4 and to a transmission line 51 of the temperature sensor 5.

The control device 6 cooperates with the temperature sensor 5 to control on/off of an electric power source 7 connected to the power cord 41, controlling the temperature of the needle 3 to be in a preset temperature range between 40° C. and 45° C. The other end 31 of the needle 31 melts and removes the lipids blocking the Meibomian gland. The operating effect of treatment of Meibomian gland dysfunction is enhanced, and the success rate is increased. The effective period of time of the treatment is significantly increased while obtaining high percentage of effective operations.

With reference to FIGS. 1-5, the housing 1 includes two half housings 11 and 12 engaged together to define the compartment 13. In the form shown, half casing 11 includes a plurality of engaging plates 111, and half housing 12 includes a plurality of engaging grooves 121 engaged with the engaging plates 111. The compartment 13 includes a battery space 15 delimited by a partitioning board 152. Batteries 71 and 72 are received in the battery space 15 and electrically connected to the heating member 4 and serve as the electric power source 7 for the heating member 4. The housing 1 further includes a lid 17 and a hinge 18 facing an opening 151 of battery space 15. The lid 17 includes a hook 171 for retaining the lid 171 in a position closing the battery space 15.

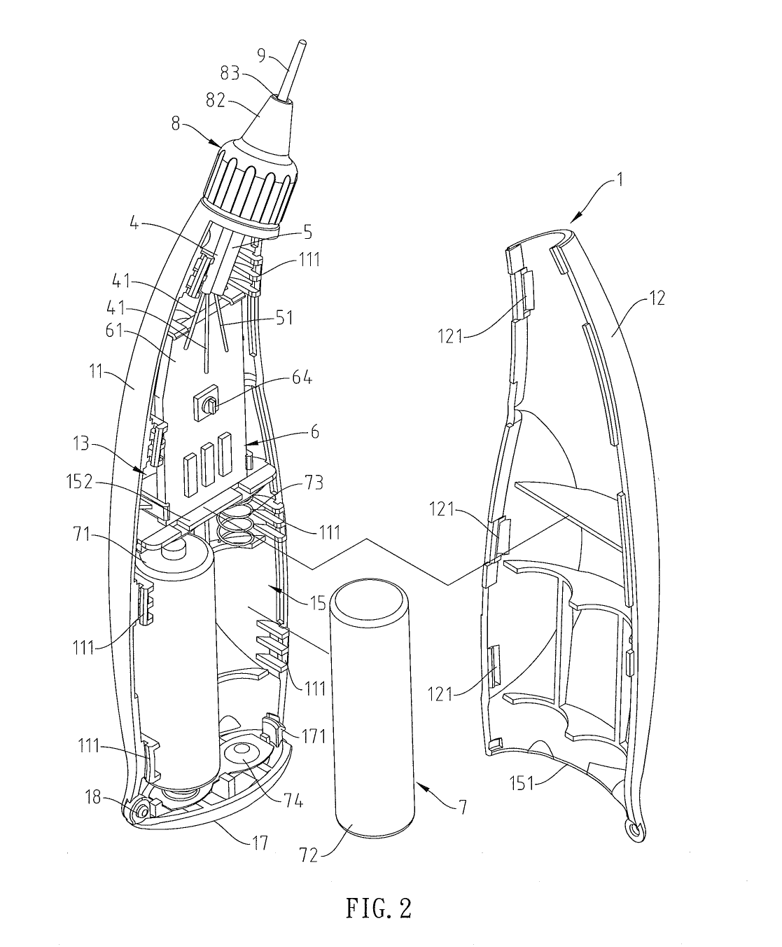

With reference to FIGS. 1 and 2, the control device 6 includes a circuit board 61 on which a switch 62 and an indicator lamp 63 are mounted. The switch 62 and the indicator lamp 63 are exposed on the outer face of the housing 1. The switch 62 controls on/off of the electric power source 7 for the heating member 4. The indicator lamp 63 indicates on/off of the electric power source 7. The control device 6 further includes a temperature adjusting member 64 on the circuit board 61. The temperature adjusting member 64 can be operated to adjust the preset temperature range of the needle 3.

With reference to FIGS. 1-5, the coupling portion 21 of the coupler 2 has a section extending out of the seat 14 of the housing 1. The section of the coupling portion 21 includes an engagement hole 23. The engagement hole 23 receives and holds the end 32 of the needle 3. The second end of the coupler 2 includes two spaced receptacles 22 and 24. The receptacles 22 and 24 respectively couple with the heating member 4 and the temperature sensor 5. A thermal insulating ring 26 is mounted around the coupler 2 and sandwiched between the coupler 2 and an inner periphery of the seat 4, positioning the coupler 2 in the seat 14 and avoiding heat from being conducted to the housing 1.

In the form shown, the needle 3 is disposable and includes an enlarged shank 33 mounted on the end 32 of the needle 3 and having an outer diameter larger than that of the end 32 of the needle 3 to allow easy mounting of the needle 3. When not in use, a protective cover 9 is mounted around the other end 31 of the needle 3 and the shank 33. The coupling portion 21 of the coupler 2 includes a conic outer periphery 25 tapering away from the second end of the coupler 2. The coupling portion 21 includes a plurality of slits 27 extending longitudinally to allow radial expansion while mounting the end 32 of the needle 3 and the shank 33. The seat 14 of the housing 1 includes an outer periphery having a threaded portion 16. A positioning cap 8 has an inner threaded portion 81 engaged with the threaded portion 16 of the seat 14. The positioning cap 8 includes a positioning portion 82 having a central conical hole 83. The central conical hole 83 has an inner periphery abutting the conic outer periphery 25 of the coupling portion 21 of the coupler 2 to hold the end 32 of the needle 3 and the shank 33 by the coupling portion 21. The other end 31 of the needle 31 extends out of the central conical hole 83. Thus, the disposable needle 3 can be easily mounted and detached to assure safety and hygiene.

Although specific embodiments have been illustrated and described, numerous modifications and variations are still possible without departing from the essence of the invention. The scope of the invention is limited by the accompanying claims.

1. A lipid removal device for treating Meibomian gland dysfunction comprising:
a housing defining a compartment, with the housing including a hollow seat formed on an end of the housing;
a thermally conductive coupler mounted in the seat of the housing, with the coupler including first and second ends, with the first end of the coupler having a coupling portion, with the coupling portion engaged with an end of a needle, with the other end of the needle adapted to be inserted into a blocked Meibomian gland of an eyelid of a human body and adapted to be heated to remove lipids blocking the Meibomian gland;
a heating member mounted in the compartment of the housing and connected to the second end of the coupler;
a temperature sensor mounted in the compartment of the housing and connected to the coupler;
a control device mounted in the compartment of the housing and electrically connected to a power cord of the heating member and to a transmission line of the temperature sensor, with the control device cooperating with the temperature sensor to control on/off of an electric power source connected to the power cord, controlling a temperature of the needle to be in a preset temperature range.

2. The lipid removal device for treating Meibomian gland dysfunction as claimed in claim 1, with the housing including two half housings engaged together to define the compartment, with the compartment including a battery space, with the electric power source being a battery received in the battery space and electrically connected to the heating member.

3. The lipid removal device for treating Meibomian gland dysfunction as claimed in claim 2, with the housing further including a lid, with the lid closing the battery space.

4. The lipid removal device for treating Meibomian gland dysfunction as claimed in claim 1, with the coupling portion of the coupler having a section extending out of the seat of the housing, with the section of the coupling portion including an engagement hole, with the engagement hole receiving and holding the end of the needle.

5. The lipid removal device for treating Meibomian gland dysfunction as claimed in claim 4, with the second end of the coupler including two spaced receptacles, with the two spaced receptacles respectively coupling with the heating member and the temperature sensor, with an thermal insulating ring mounted around the coupler and sandwiched between the coupler and an inner periphery of the seat, positioning the coupler in the seat.

6. The lipid removal device for treating Meibomian gland dysfunction as claimed in claim 4, with an enlarged shank mounted on the end of the needle and having an outer diameter larger than that of the end of the needle.

7. The lipid removal device for treating Meibomian gland dysfunction as claimed in claim 6, further comprising: a protective cover mounted around the other end and the shank of the needle.

8. The lipid removal device for treating Meibomian gland dysfunction as claimed in claim 4, with the coupling portion of the coupler including a conic outer periphery tapering away from the second end of the coupler, with the coupling portion including a plurality of slits extending longitudinally, with the seat of the housing including an outer periphery having a threaded portion, with the lipid removal device further comprising: a positioning cap having an inner threaded portion engaged with the threaded portion of the seat, with the positioning cap including a positioning portion having a central conical hole, with the central conical hole having an inner periphery abutting the conic outer periphery of the coupling portion of the coupler to hold the end of the needle by the coupling portion, with the other end of the needle extending out of the central conical hole.

9. The lipid removal device for treating Meibomian gland dysfunction as claimed in claim 1, with the control device including a switch, with the switch controlling on/off of the electric power source for the heating member.

10. The lipid removal device for treating Meibomian gland dysfunction as claimed in claim 9, with the control device including a temperature adjusting member, with the temperature adjusting member operable to adjust the preset temperature range of the needle.

11. The lipid removal device for treating Meibomian gland dysfunction as claimed in claim 1, with the preset temperature range of the needle being between 40° C. and 45° C.
